(19) United States
(12) Patent Application Publication
(10) Pub. No.: US 2009/0258955 A1
(43) Pub. Date: Oct. 15, 2009

(54) INTRAOCULAR IRRIGATING SOLUTION HAVING IMPROVED FLOW CHARACTERISTICS

(75) Inventors: Mandar V. Shah, Rockaway, NJ (US); Mikhail Boukhny, Laguna Niguel, CA (US); William H. Garner, Eastport, ME (US); Kerry L. Markwardt, Burleson, TX (US); Uday Doshi, Randolph, NJ (US)

Correspondence Address: ALCON, IP LEGAL, TB4-8, 6201 SOUTH FREEWAY, FORT WORTH, TX, 76134, US

(73) Assignee: ALCON, INC., Hunenberg (CH)

(21) Appl. No.: 12/423,727

(22) Filed: Apr. 14, 2009

Related U.S. Application Data
(63) Continuation-in-part of application No. 11/867,523, filed on Oct. 4, 2007, now abandoned.
Application No. 11/867,523 is a continuation of application No. 11/056,042, filed on Feb. 11, 2005, now abandoned.
Application No. 11/056,042 is a continuation of application No. 10/240,449, filed on Sep. 30, 2002, now Pat. No. 7,084,130.
(60) Provisional application No. 60/257,570, filed on Dec. 20, 2000.

(51) Int. Cl. A61K 47/38; A61K 47/02

(52) U.S. Cl. 514/781; 514/769

(57) ABSTRACT

Improved intraocular irrigating solutions are described comprising a cellulose derivative such as hydroxypropylmethylcellulose. In addition, certain solutions of the present invention comprise glutathione and dextrose. The solutions have enhanced viscosities that reduce the risk of damage to intraocular tissues during intraocular surgical procedures by reducing the turbulence of the solutions and dampening the movement of tissue fragments and air bubbles. The solutions preferably also have modified surface tensions that more closely resemble the surface tension of the aqueous humor.

### CROSS-REFERENCE TO RELATED APPLICATION

The present application is a continuation-in-part of U.S. application Ser. No. 11/867,523 filed Oct. 4, 2007, which is a continuation of U.S. application Ser. No. 11/056,042 filed Feb. 11, 2005, which is a continuation of a 371 application, U.S. application Ser. No. 10/240,449 filed Sep. 30, 2002, which claims benefit of International Patent Application Number PCT/US01/48094 filed Dec. 11, 2001, which claims benefit of U.S. Provisional Application Ser. No. 60/257,570, filed Dec. 20, 2000, the contents of which are hereby incorporated by reference.

### BACKGROUND OF THE INVENTION

The present invention is directed to the field of intraocular surgery. More specifically, the invention is directed to the irrigation of intraocular tissues during cataract surgery, vitrectomy surgery, and other intraocular surgical procedures. The invention provides intraocular irrigating solutions that have improved physical properties (e.g., flow characteristics) relative to prior ophthalmic irrigating solutions.

The field of intraocular surgery has advanced dramatically over the past twenty years. The advancements in this art have resulted from significant improvements in the areas of surgical techniques, surgical equipment and associated pharmaceutical products. Despite these advancements, intraocular surgery is still a very delicate process with little room for error and great potential for harm to both ocular tissues and, ultimately, the vision of the patient. Thus, there is an ongoing need to improve ophthalmic surgical techniques and equipment, as well as associated pharmaceutical products.

The present invention has resulted from an effort to improve the fluid dynamics of intraocular irrigating solutions, so as to provide greater protection for delicate intraocular tissues, while at the same time enhancing the ability of ophthalmic surgeons to perform surgical procedures more efficiently.

Although various techniques have been used previously to remove the natural crystalline lens of the eye when it becomes afflicted with a cataract, the majority of cataract surgeries today are performed by using a procedure known as “phacoemulsification”. This procedure involves the use of a surgical handpiece having a tip that vibrates at an ultrasonic frequency. The vibrating tip of the handpiece is utilized to disintegrate or “emulsify” the cataractous lens. This process necessarily generates lens fragments or particles within the eye that can cause irreparable physical damage to corneal endothelial cells if those cells are left unprotected. The corneal endothelial cells are normally protected during the phacoemulsification procedure by injecting a viscoelastic material (e.g., hyaluronic acid) into the eye to form a protective barrier over the corneal endothelial cells. However, even with the presence of the viscoelastic material, lens particles continue to move in the eye, particularly when the viscoelastic material is removed by a combined irrigating/aspiration handpiece following the phacoemulsification of the lens, prior to insertion of an artificial lens.

Due to continuous irrigation and aspiration, usually there is a lot of turbulence in the anterior chamber, within which non-aspirated lens fragments move around. In addition, the ultrasonic vibrations produced by the tip of the phacoemulsification handpiece push the lens fragments away from the tip thereby making it difficult to aspirate the fragments via the aspiration line in the tip of the handpiece. The movement of these lens fragments can cause damage to the surrounding tissue.

In addition to the lens fragments, damage may result directly from the turbulent flow of fluids intraocularly or from bubbles generated in the intraocular fluids by the phacoemulsification handpiece. Air bubbles generated during intraocular surgery have been shown to result in severe injury to the corneal endothelium in as little as twenty seconds. The turbulent flow of fluids may also cause tissue fragments to impact the delicate corneal endothelial cells or other intraocular tissues, thereby causing mechanical trauma to such tissues.

For further background regarding these problems, please refer to the following articles: Kim, et al., “Corneal endothelial damage by air bubbles during phacoemulsification”, Archives of Opthalmology, Vol. 115:81-88, 1997; Beesley et al., “The effects of prolonged phacoemulsification time on the corneal endothelium”, Annals of Opthalmology, Vol. 18(6):216-219, 1986; Kondoh et al., “Quantitative measurement of the volume of air bubbles formed during ultrasonic vibration”, Folia Opthalmogica Japan, Vol. 45(7):718-720, 1994 and Kim et al., Investigative Opthalmology & Visual Science, Vol. 37(3):S84, 1996.

The fluid dynamics of intraocular irrigating solutions is also important during vitrectomy procedures and various other types of intraocular surgical procedures. Turbulence in intraocular fluids may also result from the movements of reciprocating vitrectomy handpieces, the alternating vacuum and irrigation modes of irrigation/aspiration handpieces and movements of other surgical handpieces and devices utilized in such procedures. The elimination or reduction of such turbulence helps to protect the retina and other tissues located in the posterior segment of the eye, as well as tissues located in the anterior segment of the eye, such as the corneal endothelial cells.

In view of these potential complications, there is a need for intraocular irrigating solutions having improved physical properties that: (1) reduce the potential for turbulence within the anterior and posterior chambers of the eye, (2) help to contain the movement of tissue fragments and air bubbles within the eye, and (3) facilitate the removal of lens fragments and other tissue fragments by making it easier for the surgeon to track the fragments with the tip of the surgical handpiece. The present invention is directed to fulfilling this need. Specifically, the present invention is directed to the provision of an irrigating solution that provides for greater control of the movement of tissue fragments, air bubbles and other particles during phacoemulsification, vitrectomy and other intraocular surgical procedures. This control of particle movement is fundamentally different from the above-discussed use of a layer of viscoelastic material to protect the corneal endothelial cells by means of a cushioning effect. The irrigating solution of the present invention is designed to provide a protective effect beyond that obtained by means of viscoelastic agents.

### SUMMARY OF THE INVENTION

The present invention is directed to the provision of intraocular irrigating solutions that help to prevent the risk of damage to intraocular tissues, while facilitating the efficiency of the surgical procedures. The irrigating solutions of the present invention are low viscosity solutions that exhibit less turbulence in the presence of phacoemulsification handpieces and other intraocular surgical devices. These solutions also restrain the movement of air bubbles and tissue fragments within the eye, and generally dampen the impact of ultrasonic handpieces, liquefracture handpieces, irrigation/aspiration handpieces, microscissors, vitrectomy handpieces and other surgical devices on intraocular tissues. The restrained movement of lens fragments within the eye protects ophthalmic tissues, and facilitates a more efficient surgical procedure by enabling the ophthalmic surgeon to locate and remove lens fragments more readily.

The intraocular irrigating solutions of the present invention have a viscosity greater than that of aqueous humor, but preferably have a surface tension similar to that of aqueous humor. Existing irrigating solutions generally have a viscosity similar to that of aqueous humor, but have surface tension higher than that of aqueous humor.

The present inventors have found that a slight enhancement of the viscosity of intraocular irrigating solutions greatly improves the ability of the solutions to protect intraocular tissues by containing the movement of tissue fragments and generally reducing the turbulence of the intraocular fluids, thereby making it easier for the fragments to be tracked and removed via aspiration. This slight enhancement of irrigating solution viscosity is also beneficial in vitrectomy procedures because it reduces the pulsatile movement of the retinal tissue and limits collateral tissue damage in the eye. The reduction of pulsatile movement of retinal tissue is particularly important in cases where the retina is partially detached.

The overall performance of the irrigating solutions of the present invention can be further enhanced by including an agent which reduces the surface tension to a level comparable to that of aqueous humor, thereby making the solutions more physiological.

Another embodiment of the present invention is an improved ophthalmic irrigating solution comprising a cellulose derivative such as hydroxypropyl methylcellulose. The ophthalmic irrigating solution may comprise additional compounds such as physiological redox agents and energy sources.

### BRIEF DESCRIPTION OF THE DRAWINGS

FIG. 1 is a graph showing the effect of viscosity on flow rate;

FIG. 2 is a graph showing the relationship between HPMC concentration and accumulation rate; and

FIG. 3 is a graph showing the mean endothelial mucin layer thicknesses for corneas subjected to continuous or pulsed phacoemulsification using irrigating solutions with (NGOIS) or without HPMC (BSS PLUS®) versus a control group.

### DETAILED DESCRIPTION OF THE INVENTION

The irrigating solutions of the present invention comprise a balanced electrolyte solution and an amount of a biologically compatible viscosity-adjusting agent sufficient to enhance the viscosity of the electrolyte solution.

The electrolyte solution utilized in the present invention will typically be a balanced salt solution, such as BSS® (Balanced Salt Solution) Sterile Irrigating Solution manufactured by Alcon Laboratories, Inc., or BSS PLUS® (Balanced Salt Solution) Sterile Irrigating Solution, also manufactured by Alcon Laboratories, Inc. However, the invention is not limited relative to the types of balanced salt solutions or other electrolyte/nutrient solutions that may be utilized as a building block for the solutions of the present invention.

The agents utilized to adjust the viscosity of the electrolyte solution will comprise one or more compounds that are compatible with intraocular tissues, such as: chondroitin sulfate, sodium hyaluronate or other proteoglycans; cellulose derivatives, such as hydroxypropyl methylcellulose (“HPMC”), carboxy methylcellulose (“CMC”), and hydroxyethyl cellulose (“HEC”); collagen and modified collagens; galactomannans, such as guar gum, locust bean gum and tara gum, as well as polysaccharides derived from the foregoing natural gums and similar natural or synthetic gums containing mannose and/or galactose moieties as the main structural components (e.g., hydroxypropyl guar); xanthan gum; gellan gums; alginate; chitosans; polyvinyl alcohol; carboxyvinyl polymers (e.g., carbomers such as the Carbopol® brand polymers available from B.F. Goodrich); and various other viscous or viscoelastomeric substances, including but not limited to those described in U.S. Pat. No. 5,409,904 (Hecht, et al.), the entire contents of which are hereby incorporated by reference in the present specification.

The following patent publications may be referred to for further details concerning the above-listed viscosity-enhancing agents: U.S. Pat. No. 4,861,760 (gellan gums); U.S. Pat. No. 4,255,415 and WIPO Publication No. WO 94/10976 (polyvinyl alcohol); U.S. Pat. No. 4,271,143 (carboxyvinyl polymers); WIPO Publication No. WO 99/51273 (xanthan gum); and WIPO Publication No. WO 99/06023 (galactomannans). The entire contents of the foregoing references pertaining to the structures, chemical properties and physical properties of the respective viscosity enhancing agents described above are hereby incorporated in the present specification by reference.

The above-described viscosity-adjusting agents will be utilized in an amount sufficient to provide the irrigating solutions of the present invention with an enhanced viscosity. As utilized herein, the phrase “enhanced viscosity” means a viscosity which is greater than the viscosity of aqueous humor and prior irrigating solutions, both of which generally have viscosities of approximately 1 centipoise (“cP”) measured at 37° C. and 20° C., respectively (as shown in TABLE 5 below). The irrigating solutions of the present invention will typically have viscosities of from greater than 1 cP to about 15 cP, preferably from about 2 to about 7 cP, and most preferably about 3 cP as measured by the Ubbelohde viscometer method at 20° C. This method is described by James et al. (J. Phys. D: Appl. Phys., Vol. 17:225-230, 1984) and is also described in Example 4 below.

The amount of viscosity adjusting agent utilized will vary depending on the degree of viscosity enhancement desired and the specific agent or agents selected. However, the concentration of the viscosity-adjusting agent in the irrigating solutions of the present invention will typically range from about 0.1 to about 1.0 weight/volume percent (“w/v %”) for polymers such as HPMC.

The solutions of the present invention may also comprise a physiological redox agent such as glutathione that is used to prevent or reduce oxidative damage to corneal cells. Glutathione disulfide (GSSG) is optionally present in the solutions of the present invention at concentrations ranging from 0.001 to 0.1 w/v %. A preferred range is 0.01 to 0.05 w/v %. GSSG is most preferred at a concentration of about 0.02 w/v %.

Other solutions may comprise carbohydrate energy sources that may be utilized by corneal cells. In a preferred embodiment, an energy source is a monosaccharide such as dextrose or a polysaccharide such as sucrose. A preferred energy source concentration range is 0.01 to 0.1 w/v % and is most preferred at a concentration of about 0.09 w/v %.

Bicarbonate is a physiological buffer for the eye and bicarbonate salts are recognized as key components of ophthalmic irrigating solutions. Thus, embodiments of the present invention may include one or more bicarbonate salts at various concentrations including, without limitation, salts such as sodium or potassium bicarbonate. Preferred embodiments of the present invention comprise sodium bicarbonate. The concentration range of bicarbonate may be from about 0.1 w/v % to about 1.0 w/v % and the most preferred concentration is about 0.21 w/v %.

The solutions of the present invention may comprise additional essential ions such as sodium, potassium, and chloride. Potassium and sodium may be provided in the form of various sodium and potassium salts known to those of skill in the art, such as sodium or potassium chlorides, sulfates, acetates, citrates, lactates, and gluconates. Similarly, chloride salts, such as sodium chloride and potassium chloride, may be used to provide chloride in solutions of the present invention. For the essential ions, the concentration of potassium should be about 0.01 w/v % to about 0.5 w/v %, with the most preferred concentration about 0.04 w/v %. The concentration of sodium should be about 0.1 w/v % to about 1.0 w/v %, with the most preferred concentration about 0.7 w/v %. Other ionic salts such as calcium chloride and magnesium chloride may be included in certain embodiments. The preferred concentration of calcium chloride is about 0.01 w/v % to about 0.5 w/v %, with a most preferred concentration of about 0.015 w/v %. The preferred concentration of magnesium chloride is about 0.01 w/v % to about 0.5 w/v %, with a most preferred concentration of about 0.02 w/v %.

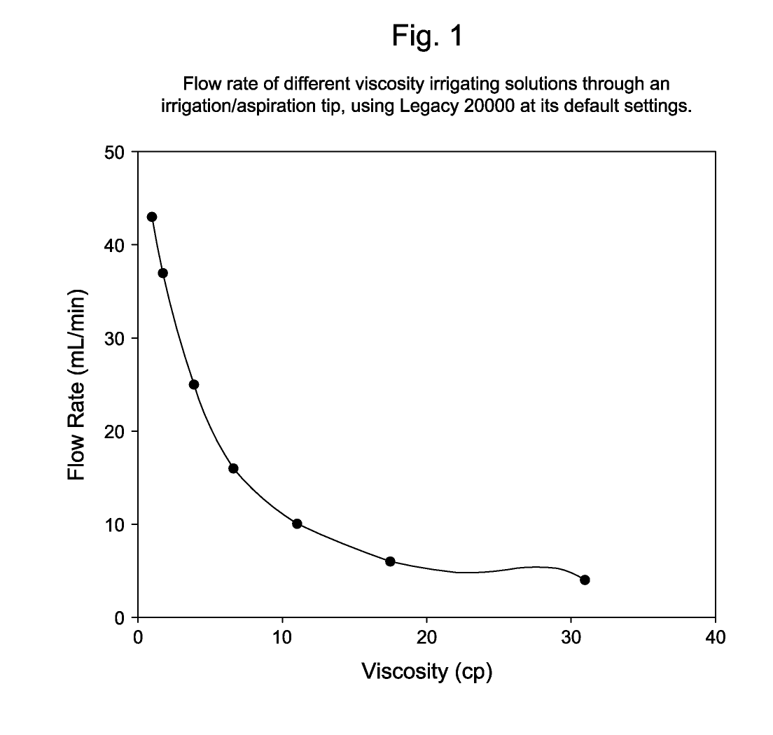

It should be noted that it is necessary to achieve a balance between: (a) enhancing the viscosity of the solution, and (b) maintaining a solution viscosity that is acceptable for use with the irrigation/aspiration system employed during intraocular surgical procedures. FIG. 1 of the accompanying drawings is a graph showing the flow rate of irrigating solutions of different viscosities through a normal irrigation/aspiration tip in the Series 20000 Legacy® (“STTL”) surgical operating system available from Alcon Laboratories, Inc. During generation of these data, all the settings on the STTL system were default instrumental settings. FIG. 1 clearly shows the effect of increasing viscosity on flow rate of the irrigating solution, which is usually flowing under gravity.

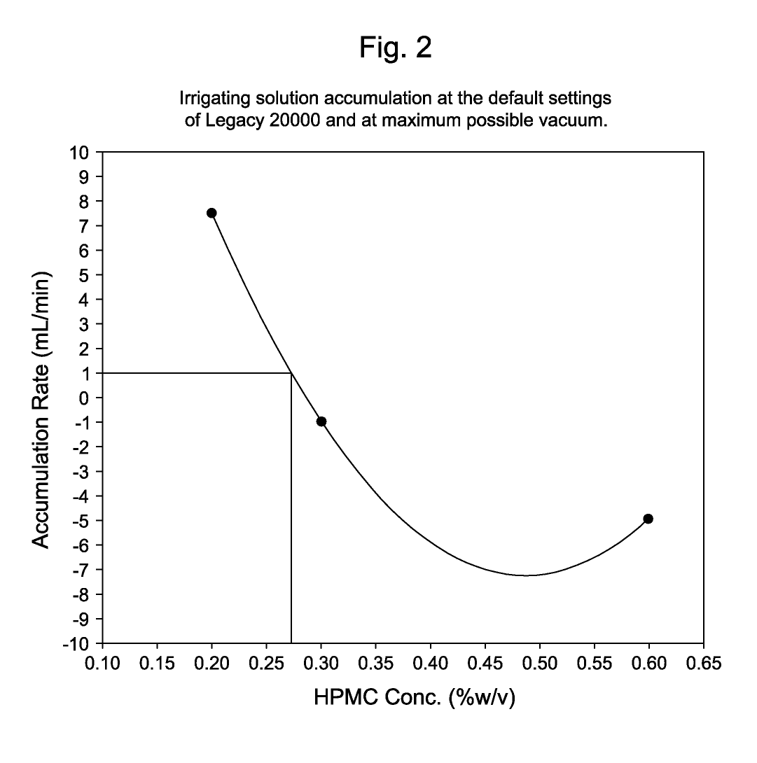

During a surgical procedure, aspiration is carried out by applying vacuum through the tip of a surgical handpiece. Generally, the maximum vacuum or suction capability of the system is such that the irrigation rate is higher than the aspiration rate to maintain positive flow. Hence, the increase in viscosity of the irrigation solution should be such that the flow rate remains greater than the maximum aspiration rate. FIG. 2 of the accompanying drawings illustrates this point.

Increasing the concentration of the viscosity-adjusting agent increases the viscosity of the solution, so at the same bottle height, the normal gravity fed irrigation flow rate of fluid into the eye decreases. As the net irrigation rate decreases, the effective aspiration rate, which is controlled independently by a peristaltic pump on the STTL, increases. Hence, the accumulation rate goes from a positive to a negative value. A minimum irrigation rate of 1 milliliter/minute of aspiration is needed to prevent drying up of the tissue. These competing factors must be balanced. In the case of HPMC, it has been determined that a HPMC concentration of 0.27 w/v % provides the desired level of viscosity enhancement without impeding normal irrigation and aspiration functions. It should be noted that this ideal concentration was determined using HPMC (E4M) in connection with the STTL surgical operating system and a standard phacoemulsification tip. The ideal concentration may vary somewhat, depending on the surgical operating system and phacoemulsification tip utilized.

The preferred viscosity-adjusting agent is hydroxypropylmethylcellulose (“HPMC”). The present inventors have found that the addition of HPMC to a conventional balanced salt solution results in a significant reduction in turbulence during intraocular surgery, relative to the turbulence seen with the balanced salt solution alone. The preferred concentration of HPMC is about 0.2 to 0.3 w/v %, but this range may vary slightly depending on the particular ophthalmic surgical system being utilized and the instrument settings of that system. Irrigating solutions containing this concentration of HPMC will have a viscosity of about 4 to 6 cP. The most preferred viscosity-adjusting agent is HPMC (E4M) at a concentration of 0.22 to 0.27 w/v %.

As indicated above, the irrigating solutions of the present invention preferably also include an agent to modify the surface tension of the solutions so as to resemble the surface tension of the aqueous humor. The surface tension of the aqueous humor is approximately 60 dynes per centimeter or milliNewtons per meter (“mN/m”) as measured by the pendant drop method at 23° C. and further described in Example 4 below. The irrigating solutions of the present invention will therefore preferably have a surface tension in the range of 40 to 60 mN/m as measured by the pendant drop method at 23° C. as reported by Ghate et al. (Cornea, Vol. 27(9):1050-1056, 2008), and further described in Example 4 below.

It should be noted here that viscosity can be increased by an appropriate agent without affecting surface tension, and that surface tension can be reduced to the level of aqueous/vitreous humor by inclusion of an appropriate surface-active agent independent of viscosity. Thus, these two physical properties of irrigating solutions are independent of each other. However, in some cases, the viscosity-adjusting agent may also function as the surface tension reducing agent. This is true with respect to the preferred embodiment of the present invention, wherein HPMC is utilized both as a viscosity-adjusting agent and a surface tension reducing agent.

In other cases, it may be necessary to add a separate agent to the irrigating solution for purposes of reducing the surface tension of the solution. Possible agents which can be utilized for this purpose include: Polyoxyl 35 castor oil (Cremophor® EL and Cremophor® EL-P, available from BASF Corp.), Polyoxyl 40 Hydrogenated Castor Oil (HCO-40), Solutol® HS 15 (BASF Corp.), Polysorbate 80, Tocophersolan (TPGS), and other ophthalmically acceptable surface active agents.

The following examples are provided to further illustrate various features of the present invention.

### Example 1

| Component | Amount (w/v %) | Function |
|---|---|---|
| HPMC (E4M) | 0.1 to 0.3 | Viscosity |
| | | and Surface |
| | | Tension Modifier |
| Sodium Chloride | 0.744 | Tonicity Agent |
| Potassium Chloride | 0.0395 | Essential Ion |
| Dibasic Sodium Phosphate | 0.0433 | Buffering Agent |
| (Anhydrous) | | |
| Sodium Bicarbonate | 0.219% + 20% xs | Physiological |
| | | Buffer |
| Hydrochloric Acid | Adjust pH | pH Adjust |
| Sodium Hydroxide | Adjust pH | pH Adjust |
| Water for Injection | 100% | Vehicle |

The above-described formulation may be prepared as follows: First, the water for Injection is brought close to boiling or at boiling. The HPMC is then slowly added to the water under continuous stirring to thoroughly disperse it in the water. Then the mixture is slowly allowed to cool, stirring continuously. Once at room temperature, the mixture should start clearing up. Then the mixture is stored overnight in an appropriate container to fully hydrate the HPMC. The following day, the remaining ingredients are added to the HPMC solution, additional water for injection is added if needed to bring the solution to final volume, and the final solution is filtered, packaged in bottles and autoclaved.

### Example 2

| Component | Amount (w/v %) | Function |
|---|---|---|
| HPMC (E4M) | 0.1 to 0.3 | Viscosity |
| | | and Surface |
| | | Tension Modifier |
| Sodium Chloride | 0.64 | Tonicity Agent |
| Potassium Chloride | 0.075 | Essential Ion |
| Calcium Chloride (Dihydrate) | 0.048 | Essential Ion |
| Magnesium Chloride | 0.03 | Essential Ion |
| (Hexahydrate) | | |
| Sodium Acetate (Trihydrate) | 0.039 | Buffering Agent |
| Sodium Citrate (Dihydrate) | 0.17 | Buffering Agent |
| Hydrochloric Acid | Adjust pH | pH Adjust |
| Sodium Hydroxide | Adjust pH | pH Adjust |
| Water for Injection | 100% | Vehicle |

The above-described formulation may be prepared by means of the method described in Example 1, above. The physical parameters for this formulation are presented in TABLE 1, below.

### Example 3

Three solutions were prepared and tested to evaluate the physical properties of the solutions of the present invention versus related solutions. The solutions tested and the respective physical properties of the solutions were as follows in TABLE 1. The physical property measurements of TABLE 1 were made as described in Example 4.

TABLE 1

| | Osmolality | Viscosity | Surface Tension |
|---|---|---|---|
| Solution | mOsm/kg | (cP) | mN/m |
| BSS ®* | 304, 305 | 1.02, 1.06 | 70, 73 |
| BSS ® + 0.05% | 305, 305 | 0.99, 1.01 | 43, 43 |
| Cremophor ® EL | | | |
| BSS ® + 0.3% | 320, 322 | 6.9, 7.0 | 48, 49 |
| HPMC (grade | | | |
| E4M) (Example 2 | | | |
| Formulation) | | | |

*As utilized in Table 1, the term “BSS ®” refers to BSS ® (Balanced Salt Solution) Sterile Irrigating solution manufactured by Alcon Laboratories, Inc., Fort Worth, Texas.

As indicated above, the addition of 0.3% HPMC to the BSS solution increased the viscosity from approximately 1 cps to 7 cP, and reduced the surface tension from approximately 71.5 mN/m to approximately 48.5 mN/m. Thus, the addition of this amount of HPMC increased the viscosity of the balanced salt solution and reduced its surface tension, in accordance with the basic principles of the present invention. Conversely, the addition of 0.05% Cremophor® EL to the balanced salt solution had no effect on viscosity, but reduced the surface tension of the balanced salt solution from approximately 71.5 mN/m to 43 mN/m.

The above-identified solutions were tested in a simulated intraocular surgery model to determine if the addition of Cremophor® EL and HPMC to the balanced salt solution affected the performance of the solution relative to the turbulence of the solution during intraocular surgical procedures. It was determined that the addition of Cremophor to the balanced salt solution, although effective in reducing the surface tension of the solution, had little if any effect on the performance of the balanced salt solution. However, the solution containing HPMC demonstrated much less turbulence than the balanced salt solution alone. This turbulence was judged based on the movement of air bubbles and the movement of lens fragments.

The spinning and rotation of lens fragments seen with the balanced salt solution alone was reduced significantly by the inclusion of HPMC in the solution. The dampening of the movement of the lens particles facilitated an easier removal of the particles from the eye during the simulated surgical procedure. This dampening effect facilitated a more efficient surgical procedure and reduced the time required for the procedure.

Conversely, there appeared to be no difference between the balanced salt solution alone and the balanced salt solution containing cremophor with regard to bubble formulation, rate of flow or the visual hydrodynamics of the irrigating solutions.

The foregoing results confirm that the addition of a small amount of a viscosity enhancing agent reduces the turbulence of intraocular fluids during surgical procedures, dampens the movement of bubbles and lens fragments, and generally renders the procedure more efficient.

### Example 4

A two-part ophthalmic irrigating solution denoted hereinafter as NGOIS, was prepared and tested to evaluate the physical properties of the solutions of the present invention versus related solutions. The two parts of the solution are reconstituted prior to use in the proportions 480 mL of NGOIS Part 1 to 20 mL of NGOIS Part II. The compositions of the Part I and Part II portions of BSS PLUS® and NGOIS are presented in TABLES 2-3. The reconstituted solution compositions are presented in TABLE 4. The respective physical properties of reconstituted BSS PLUS®, reconstituted NGOIS and human aqueous humor are presented in TABLE 5.

TABLE 2

| | BSS PLUS ® ^{a} | NGOIS | |
|---|---|---|---|
| | Part I | Part I | |
| Component | (w/v %) | (w/v %) | Function |
| HPMC (E4M) | — | 0.13 to 0.18 | Viscosity and |
| | | | Surface Tension |
| | | | Modifier |
| Sodium Chloride | 0.744 | 0.744 | Tonicity Agent |
| Potassium Chloride | 0.0395 | 0.0395 | Essential Ion |
| Dibasic Sodium | 0.0433 | 0.0433 | Buffering Agent |
| Phosphate | | | |
| (Anhydrous) | | | |
| Sodium Bicarbonate | 0.219% + | 0.219% + | Physiological |
| | 20% xs | 20% xs | Buffer |
| Hydrochloric Acid | Adjust pH | Adjust pH | pH Adjust |
| Sodium Hydroxide | Adjust pH | Adjust pH | pH Adjust |
| Water for Injection | 100% | 100% | Vehicle |
| Volume | 480 mL | 480 mL | |

^{a} As utilized in TABLES 2-5, the term “BSS PLUS ®” refers to BSS PLUS ® Sterile Intraocular Irrigating Solution (balanced salt solution enriched with bicarbonate, dextrose, and glutathione) manufactured by Alcon Laboratories, Inc., Fort Worth, Texas.

TABLE 3

| | BSS PLUS ® | NGOIS | |
|---|---|---|---|
| | Part II | Part II | |
| Component | (w/v %) | (w/v %) | Function |
| Glutathione | 0.46 + 25% | 0.46 + 25% | Physiological |
| Disulfide | excess | excess | Redox Agent |
| Calcium Chloride | 0.385 | 0.385 | Essential Ion |
| (Dihydrate) | | | |
| Magnesium | 0.5 | 0.5 | Essential Ion |
| Chloride | | | |
| (Hexahydrate) | | | |
| Dextrose | 2.3 | 2.3 | Physiological |
| (Anhydrous) | | | Energy Source |
| Water for Injection | 100% | 100% | Vehicle |
| Volume | 20 mL | 20 mL | |

TABLE 4

| | Reconstituted | Reconstituted | |
|---|---|---|---|
| | BSS PLUS ® | NGOIS | |
| Component | (w/v %) | (w/v %) | Function |
| HPMC (E4M) | — | 0.125-0.173 | Viscosity and |
| | | | Surface Tension |
| | | | Modifier |
| Glutathione | 0.0184 | 0.0184 | Physiological |
| Disulfide | | | Redox Agent |
| Sodium Chloride | 0.714 | 0.714 | Tonicity Agent |
| Potassium Chloride | 0.038 | 0.038 | Essential Ion |
| Calcium Chloride | 0.0154 | 0.0154 | Essential Ion |
| (Dihydrate) | | | |
| Magnesium | 0.02 | 0.02 | Essential Ion |
| Chloride | | | |
| (Hexahydrate) | | | |
| Dextrose | 0.092 | 0.092 | Physiological |
| (Anhydrous) | | | Energy Source |
| Dibasic Sodium | 0.042 | 0.042 | Buffering Agent |
| Phosphate | | | |
| (Anhydrous) | | | |
| Sodium Bicarbonate | 0.21 | 0.21 | Physiological |
| | | | Buffer |
| Hydrochloric Acid | Adjust pH | Adjust pH | pH Adjust |
| Sodium Hydroxide | Adjust pH | Adjust pH | pH Adjust |
| Water for Injection | 100% | 100% | Vehicle |
| Volume | 500 mL | 500 mL | |

The physical property measurements presented in TABLES 1 & 5 were determined according to the following methods. Viscosity determinations were made at controlled temperatures using either an Ubbelohde capillary or Brookfield rotational viscometer. The Ubbelohde viscometer, a capillary-type viscometer, is a glass U-shaped tube held vertically in a controlled temperature bath. One arm has a vertical section with a precise narrow bore (the capillary), above which is a bulb. The test solution is drawn up into the upper bulb by suction and allowed to flow down past an upper graduation mark through the capillary past the lower graduation mark. The time required for the solution to flow from the top graduation line to the bottom graduation line is measured and compared to the time required for a standard liquid of known viscosity. This time is proportional to the kinematic viscosity. The viscosity of water using an Ubbelohde viscometer was reported to be 1.002 cP at 20° C. and 0.719 cP at 35° C. by James et al. (J. Phys D: Appl Phys, Vol. 17:225-230, 984).

The Brookfield viscometer is a cone-and-plate-type rotational viscometer. It measures the torque required to rotate a very shallow angle cone immersed in the test solution above a flat plate at a known speed. The sample cup, which is fitted to a controlled temperature recirculating bath, is filled with the test solution and attached to the viscometer, immersing the cone in the test solution. The cone is rotated at a known speed and the torque required to turn the cone in the test solution (the resistance to movement of the rotating cone) is a function of the viscosity of the test solution.

Surface tension determinations were made by the Wilhelmy plate, Du Nöuy ring or pendant drop methods. The Wilhelmy plate method consists of immersing a vertical plate in the test solution and measuring the maximum force exerted on the plate as it is lifted from the solution. Similarly, the Du Nouy ring method involves immersing a ring in the test solution and measuring the maximum force as the ring is lifted from the solution. Surface tension determinations by the pendant drop method are made by suspending drops of the test solution from a cannula tip and capturing images of the drops with a high speed video camera. The images are processed and analyzed to determine the drop shapes and dimensions. The drop dimensions are fitted to the Laplace-Young equation which describes the relationship between the curvature of a surface, the difference in pressure on the two sides of the surface and the surface tension. Solving the Laplace-Young equation provides the calculated surface tension.

TABLE 5

| Fluid | Viscosity ^{a} | Surface Tension ^{b} |
|---|---|---|
| Reconstituted BSS PLUS ® | 1.11 ± 0.02 cP (n = 6) | 72.18 ± 0.68 mN/m (n = 5) |
| Reconstituted NGOIS | 3.16 ± 0.08 cP (n = 8) | 49.57 ± 2.41 mN/m (n = 5) |
| Human Aqueous Humor | 1.00 ± 0.01 (n = 5) ^{c} | 59.16 ± 2.38 mN/m (n = 7) |

^{a} Ubbelohde viscometer, 20° C.
^{b} Pendant drop method at 23° C.; as reported by Ghate et al. (Cornea, Vol. 27(9): 1050-1056, 2008).
^{c} Relative viscosity, Ostwald viscometer at 37° C. as reported by Beswick et al. (British J. Ophthal, Vol. 40(9): 545-548, 1956).

### Example 5

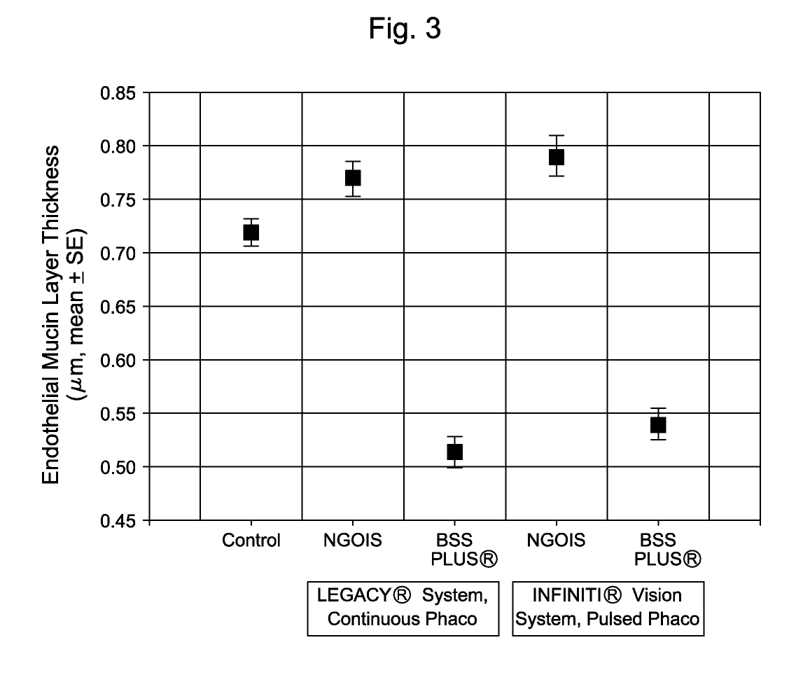

The endothelial surface of the cornea is protected by the mucin layer, a coating that forms a physical barrier to stressors such as osmotic and pH variations and that can be damaged during cataract surgery. The thickness of the mucin layer is a measure of its protective capacity, and in vitro human corneal studies were conducted by Ghate et al. (Cornea, Vol. 27(9):1050-1056, 2008) to evaluate endothelial mucin layer thickness and endothelial ultrastructure after either continuous (Alcon INFINITI® Vision System) or pulsed (Alcon SERIES 20000® LEGACY® surgical system) phacoemulsification (“phaco”) followed by irrigation/aspiration (“I/A”) with either NGOIS or BSS PLUS®. All human corneas exposed to phaco and I/A in the studies were observed to have normal endothelial ultrastructures. The corneal endothelial mucin layer thickness was 0.77±0.02 μm (mean±SE) with NGOIS and 0.51±0.01 μm with BSS PLUS® in the continuous phaco group, 0.79±0.02 μm with NGOIS and 0.54±0.01 μm with BSS PLUS® in the pulsed phaco group and 0.72±0.02 μm in the control group. The results are presented in FIG. 3.

In the continuous phaco group, the mean mucin layer thickness of the corneas irrigated with NGOIS was 51% greater than that of the corneas irrigated with BSS PLUS® (p<0.001) and 6.9% greater than that of the control corneas (p<0.015), while the mean mucin layer thickness of the corneas irrigated with BSS PLUS® was 29% less than that of the control corneas (p<0.001). In the pulsed phaco group, the mean mucin layer thickness of the corneas irrigated with NGOIS was 46% greater than that of the corneas irrigated with BSS PLUS® (p<0.001) and 9.7% greater than that of the control corneas (p<0.002), while the mean mucin layer thickness of the corneas irrigated with BSS PLUS® was 25% less than that of the control corneas (p<0.001).

NGOIS with the hydroxypropylmethylcellulose component provided better protection of the corneal endothelial mucin layer than did BSS PLUS®, as indicated by the greater average mucin layer thickness after phacoemulsification with NGOIS than with BSS PLUS®. NGOIS was also able to maintain the mucin layer thickness relative to the control corneas.

### ADDITIONAL REFERENCES

The following publications are incorporated herein by reference in their entirety:
Beswick J A, McCulloch C. Effect of Hyaluronidase on the Viscosity of the Aqueous Humor. Brit J Ophthal, Vol. 40(9):545-548, September 1956.
Ghate D A, Holley G, Dollinger H, Bullock J P, Markwardt K, Edelhauser H F. Evaluation of Endothelial Mucin Layer Thickness After Phacoemulsification With Next Generation Ophthalmic Irrigating Solution. Cornea, Vol. 27(9):1050-1056, October 2008.
James C J, Mulcahy D E, Steel B J. Viscometer calibration standards: viscosities of water between 0 and 60° C. and of selected aqueous sucrose solutions at 25° C. from measurements with a flared capillary viscometer. J Phys D: Appl Phys Vol. 17:225-230, 1984.

We claim:

1. An improved intraocular irrigating solution comprising:
hydroxypropylmethylcellulose at a concentration of 0.1 to 0.3 w/v %.

2. An improved irrigating solution according to claim 1, wherein the solution further comprises:
a physiological redox agent and an energy source.

3. An improved irrigating solution according to claim 2 wherein the physiological redox agent is glutathione and the energy source is dextrose.

4. An improved irrigating solution according to claim 3 wherein glutathione is present at a concentration of about 0.01 to 0.05 w/v % and dextrose is present at a concentration of about 0.01 to 0.1 w/v %.

5. An improved irrigating solution according to claim 4 wherein glutathione is present at a concentration of about 0.02 w/v % and dextrose is present at a concentration of about 0.09 w/v %.

6. An improved irrigating solution according to claim 1, wherein the solution has a surface tension of 40 to 60 mN/m.

7. An improved irrigating solution according to claim 6, wherein the solution has a surface tension of about 50 mN/m.

8. An improved irrigating solution according to claim 1, wherein the solution has a viscosity of from 2 to 7 cP.

9. An improved irrigating solution according to claim 8, wherein the solution has a viscosity of about 3 cP.

10. An improved irrigating solution according to claim 1, further comprising a compound selected from the group consisting of sodium chloride, potassium chloride, calcium chloride, magnesium chloride, sodium phosphate, sodium bicarbonate, and combinations thereof.

11. An improved irrigating solution according to claim 1, further comprising a surface tension modifying agent in an amount sufficient to provide the solution with a surface tension in the range of 40 to 60 mN/m.

12. A method of irrigating intraocular tissues during an ophthalmic surgical procedure, which comprises bathing the intraocular tissue with an irrigating solution according to claim 1, whereby the turbulence of the solution during the surgical procedure is reduced.

13. A method according to claim 12 wherein the solution further comprises:
a physiological redox agent and an energy source.

14. A method according to claim 13 wherein the physiological redox agent is glutathione and the energy source is dextrose.

15. A method according to claim 14 wherein glutathione is present at a concentration of about 0.01 to 0.05 w/v % and dextrose is present at a concentration of about 0.01 to 0.1 w/v %.

16. A method according to claim 15 wherein glutathione is present at a concentration of about 0.02 w/v % and dextrose is present at a concentration of about 0.09 w/v %.

17. A method according to claim 12, wherein the solution has a surface tension of 40 to 60 mN/m.

18. A method according to claim 17, wherein the solution has a surface tension of about 50 mN/m.

19. A method according to claim 12, wherein the solution has a viscosity of from 2 to 7 cP.

20. A method according to claim 19, wherein the solution has a viscosity of about 3 cP.
